(19) United States
(12) Patent Application Publication
(10) Pub. No.: US 2009/0082661 A1
(43) Pub. Date: Mar. 26, 2009

(54) SYSTEM AND METHOD TO AUTOMATICALLY ASSIST MOBILE IMAGE ACQUISITION

(75) Inventors: Jean-Pierre Saladin, Bagneux (FR); Bernard Bouvier, Eragny Sur Oise (FR); Vincent Croulard, Jouy Le Moutier (FR)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/858,570

(22) Filed: Sep. 20, 2007

(51) Int. Cl. A61B 5/00

(52) U.S. Cl. 600/415

(57) ABSTRACT

A system to generate a radiological image of a subject from multiple directions is provided. The system comprises a first drive to move the gantry and detector about a vertical axis, a second drive to move the gantry and detector about a horizontal axis, and a handle assembly coupled to the gantry in mobile support of the detector. The handle assembly includes a sensor operable to generate a signal representative of a direction of deflection of a header. The system also includes a controller having instructions generally representative of the steps that include translating the signal from the sensor in the direction of deflection of the header, generating a signal to instruct the first and second drives to move the gantry in the direction of deflection, and moving the gantry in the direction of deflection of the header via the first and second drives in accordance to the signal.

### BACKGROUND OF THE INVENTION

The subject matter herein relates generally to image acquisition, and more specifically, to a method and arrangement to assist image acquisition of a subject. Although the foregoing description refers to medical imaging, the system is also applicable to industrial imaging.

Medical imaging systems encompass a variety of imaging modalities, such as x-ray systems, computerized tomography (CT) systems, ultrasound systems, electron beam tomography (EBT) systems, magnetic resonance (MR) systems, and the like. Medical imaging systems generate images of an object, such as a patient, for example, through exposure to an energy source, such as x-rays passing through a patient. The generated images may be used for many purposes. For instance, internal defects in an object may be detected. Additionally, changes in internal structure or alignment may be determined. Fluid flow within an object may also be represented. Furthermore, the image may show the presence or absence of objects in the patient. The information gained from medical diagnostic imaging has applications in many fields, including medicine and manufacturing.

A certain conventional medical imaging system includes a mobile C-arm system. The mobile C-arm system can be used for general surgery, vascular procedures, and cardiac procedures, for example. The conventional mobile C-arm system is equipped with a radiological source or transmitter in opposed relation to a radiological detector (e.g., an image intensifier), and both are moved in relation to the imaged subject. With the subject positioned between the radiological source and detector, the C-arm system is moved and rotated so as to pass radiation through the imaged subject from various directions. As the radiation passes through the subject, anatomical structures cause variable attenuation of the radiation passing through the imaged subject and received at the detector. The detector translates the attenuated radiation into an image employed in diagnostic evaluations. In typical medical procedures around such imaging systems, multiple physicians, nurses, and technicians are located in close proximity to the imaged subject.

### BRIEF DESCRIPTION OF THE INVENTION

There is a need for an imaging system with enhanced mobility that can be readily put in arbitrary positions in a crowded work environment. The imaging system should include a power assist system operable to change a position of the imaged subject with respect to he image detector with less effort in accordance to a measured effort applied by an operator. Transition of the position of the imaged subject with respect to the image detector should be smooth and with minimum effort by the operator. The power assist system should also include an enable or kill switch that requires engagement to prevent uncontrolled motion of the subject or image detector with respect to one another. The above-mentioned needs are addressed by the embodiments described herein in the following description.

According to one embodiment, a method to assist movement of a tabletop in support of a subject to be imaged with an image acquisition system is provided. The method comprises the acts of detecting and generating an electrical signal representative of a direction of deflection of header of a control handle assembly coupled to the tabletop; interrupting communication of the electrical signal to a controller; communicating the electrical signal to a controller in response to detecting compression of the header of the control handle assembly; generating a control signal at the controller to instruct at least one of a first and a second drive to move the tabletop in the direction of deflection of the header, and moving the tabletop in the direction of deflection of the header of the control handle assembly via the at least one of the first and second drives in accordance to the control signal.

According to yet another embodiment, a system having a radiation source and a detector to generate a radiological image of a subject from multiple directions is provided. The source and detector are supported from a gantry. The system comprises a first drive coupled to move the gantry and the detector about a generally vertically aligned first axis, a second drive coupled to move the gantry and the detector about a generally horizontal aligned axis, and a control handle assembly coupled to the gantry in mobile support of the detector. The control handle assembly includes a sensor operable to generate a signal representative of a direction of deflection of a header from a neutral position. The system also includes a controller in communication with the first, second and third drives and the control handle assembly. The controller includes a processor in communication to execute a plurality of program instructions stored in a memory. The plurality of program instructions are representative of the steps that include translating the signal from the sensor into the direction of deflection of the header, generating a control signal to instruct at least one of the first and second drives to move the gantry in the direction of deflection, and moving the gantry in the direction of deflection of the header via the at least one of the first and second drives in accordance to the control signal.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a schematic diagram of an embodiment of a system that comprises an imaging system in combination with a power assist system operable to acquire medical images of a subject.

FIG. 2 shows a schematic diagram of an embodiment of the power assist system in combination with a series of drive mechanisms to move the direction of imaging by the imaging system of FIG. 1.

FIG. 3 shows a schematic diagram of a cross-section view of an embodiment of the power assist handle of the power assist system shown in FIG. 2.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

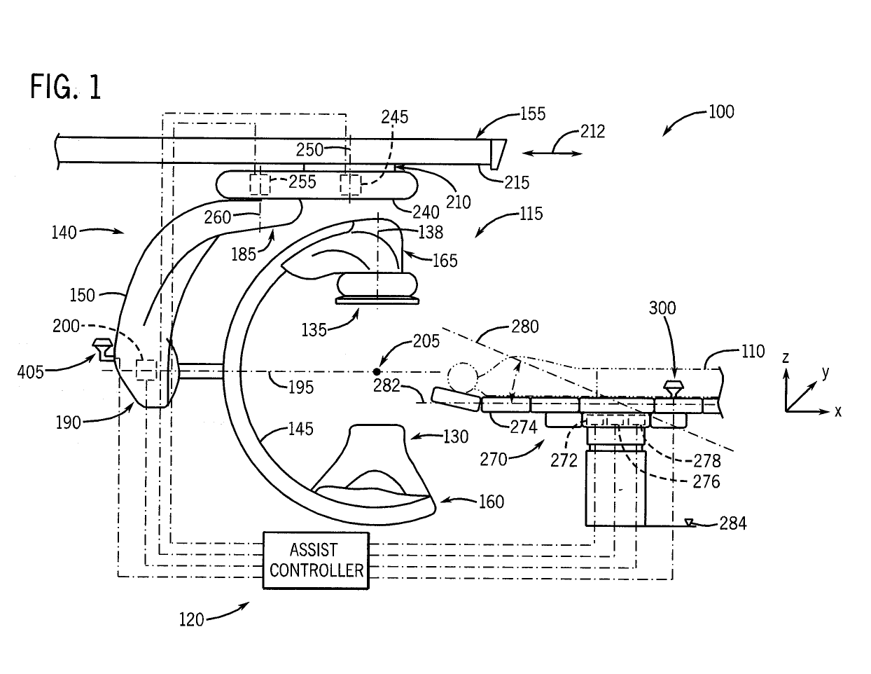

FIG. 1 illustrates an embodiment of a system 100 operable to acquire images of an imaged subject 110 from various directions. The imaged subject 110 is typically a patient or some portion of the patient 110. The system 100 generally includes an imaging system 115 in combination with a power assist system 120.

An embodiment of the imaging system 115 is a mobile X-ray imaging system operable to pass X-rays through the subject 110 and then detect and process acquired image data for interpretation. Yet, the type of imaging system 115 (e.g., computerized tomography (CT), ultrasound (US), electron beam tomography (EBT), magnetic resonance (MR), positron electron emission (PET), etc.) can vary. The illustrated embodiment of the imaging system 115 generally includes a radiation source 130 and a radiation detector or receiver 135. The radiation source 130 generates X-ray photons preferably directed to pass through a collimator and form an x-ray beam. The X-ray beam has an axis 138 that is substantially aligned with the center of the active area of the detector 135. The imaging system 115 can be operated remotely, for example so that an operator can be shielded from the radiation. Alternatively, the imaging system 115 can be placed in an examining or operating room so that a health care provider can view acquired image data while performing a medical procedure of the subject 110.

The imaging system 115 further includes a support assembly or gantry 140 in mobile support of the both the radiation source 130 and detector 135 in relation to the imaged subject 110. The support assembly 140 includes a mobile arm 145, supported by an elbow 150 from a mobile carriage system 155. The illustrated mobile arm 145 is a C-arm having a pair of opposed free ends 160 and 165. The radiation source 130 is connected at the first free end 160, and the radiation detector 135 is connected at the second free end 165 in opposed so as to receive attenuated radiation passing through the imaged subject 110 located therebetween. Yet, the shape of the mobile arm 145 can be curvilinear, angular, circular or O-shaped, etc. and is not limiting on the subject matter described herein. Examples of the mobile arm 145 include the C-arm as manufactured by GENERAL ELECTRIC® Corporation, the mobile C-arm as manufactured by Ziehm Imaging Incorporated, and the O-ARM® as manufactured by MEDTRONIC® Inc.

The elbow 150 is connected in pivotal support of the collar assembly 170 and the mobile arm 145. The illustrated elbow 150 is generally L-shaped, and includes an upper or first free end 185 and lower or second free end 190. The lower free end 190 of the elbow 150 is connected in rotational support of the collar assembly 170 and mobile arm 145 so as to rotate about an axis of rotation 195. Although the illustrated elbow 150 is L-shaped, the shape of the elbow 150 can vary. A motorized drive 200 is generally operable to rotate the mobile arm 145 and collar assembly 170 about the axis of rotation 195 with respect to the elbow 150. The support assembly 140 is configured such that the axis of rotation 195 of the mobile arm 145 intersects the axis 138 at a point of intersection, herein referred to as the isocenter 205.

The mobile carriage system 155 generally includes a carriage 210 operable to move the collar assembly 170 and mobile arm 145 in a linear direction 212 along a rail support structure 215. One embodiment of the rail support structure 215 includes a pair of parallel-aligned rails fixed overhead (e.g., to a ceiling) and configured to receive the carriage 210 therebetween. Yet, the type of rail support structure 215 and carriage 210 can vary.

An offset arm 240 is connected in rotationally support of the elbow 150 and mobile arm 145 from the mobile carriage system 155. A motorized swivel drive 245 is connected to rotate the offset arm 240 about a first axis 250 with respect to the carriage 210. An elbow drive 255 is connected to rotate the elbow 150 about a second axis 260 with the respect to the offset arm 240. The first and second axes 250 and 260 are both generally vertically aligned in parallel with one another, as well as horizontally spaced or offset a distance from one another.

The system 100 further includes a table 270 generally configured to receive the imaged subject 110. An embodiment of the table 270 comprises a first drive mechanism 272 (e.g., electric motor) operable to move a tabletop 274 in a lateral direction or x-direction, and a second drive mechanism 276 operable to move the tabletop 274 in a longitudinal direction or y-direction, in a direction generally perpendicular to the direction of the first drive mechanism 272. In combination, the first and second drive mechanisms 272, 276 are operable to move the tabletop 274 in any direction along a generally horizontal x-y plane. A third drive mechanism 278 is operable to lift the tabletop in a generally vertical direction or to an angled, slant position (as represented by dashed line and reference 280) from general horizontal 282. Yet, the system 100 can include various types of drive mechanisms (e.g., conventional advance/withdraw mechanisms, lift mechanisms and/or tilt mechanisms, etc.) operable to move a tabletop 274 in support of the imaged subject 110 to a desired raised/lowered, tilted, and/or advanced/withdrawn position. Alternatively, the tabletop 274 can be fixed with respect to a floor 284.

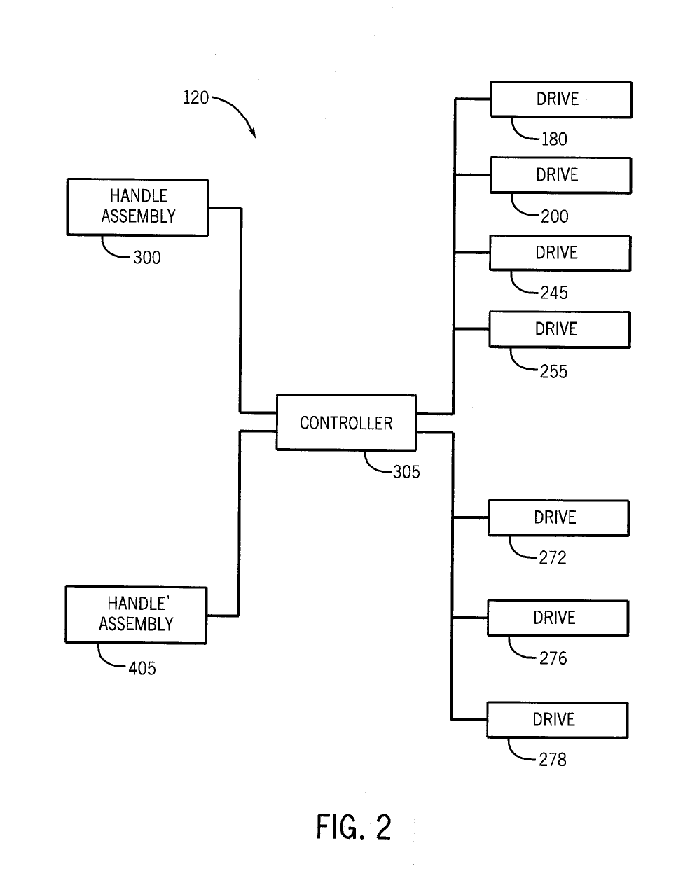

FIG. 2 illustrates one embodiment of the power assist system 120 operable in combination with the support assembly or the table 270 to change a direction of imaging of the imaged subject 110. The embodiment of the power assist system 120 generally includes a control handle assembly 300 in communication with the series of drive mechanisms 180, 200, 245, 255, 272, 276 and 278 via a controller 305. The number and type of drive mechanisms 180, 200, 245, 255, 272, 276 and 278 can vary.

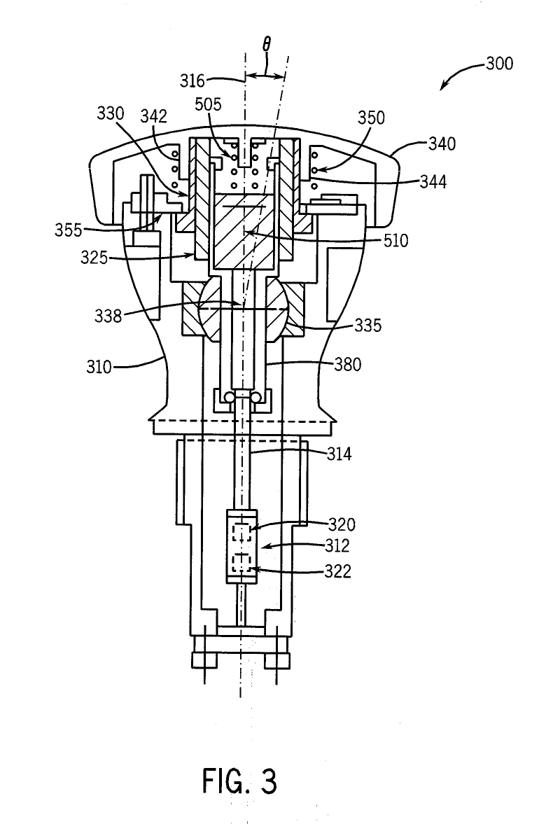

Referring to FIG. 3, an embodiment of the control handle assembly 300 generally includes a housing 310 fixed with respect to the apparatus (e.g., the tabletop 274, the gantry 140, kart, etc.) to be moved. The housing 310 generally encloses a sensor 312. The sensor 312 generally includes a stem 314 having a first end fixed or coupled to the housing 310 and a second end opposite the first end. The stem 314 is generally elongated along a longitudinal axis 316 of the control handle assembly 300. One or more strain gauges 320, 322 are attached to measure a force or strain exerted on the stem 314. For example, the first strain gauge 320 can be attached to measure a strain exerted on the stem 314 on a longitudinal direction, and a second strain gauge 320 operable to measure a strain exerted on the stem 314 in a lateral direction, generally perpendicular to the longitudinal directional force measured by the first strain gauge 320. Of course, the direction of measurement can vary.

The second end of the stem 314 is slidingly coupled to a needle bearing 325. A bushing 330 is generally coupled and fixed with respect to a radially outward surface of the needle bearing, relative to the axis 316. The needle bearing 325 generally extends through a ball pivot 335 rotatably mounted in the housing 310. The bushing 330 and needle bearing 325 are generally pivotally supported to swivel about the ball pivot 335 with respect to the housing 310 about a point 338.

A header 340 is coupled at an upper end of the bushing 330 and needle bearing 325. The header 340 generally includes a structural leg 342 that is generally cylindrical-shaped to receive the bushing 330 and needle bearing 325 fitted therein, coupled with or without an adhesive (e.g., glue). The shape and number of the leg 342 can vary. The lower end of the needle bearing 325 is coupled to a lever 380. The lever 380 is coupled to transfer or transmit angulated force at the header 340 to the stem 314 of the sensor 312. An embodiment of the lever 380 includes a hollow inner cylindrical core configured to slidably receive the stem 314 of the sensor 312 therethrough generally along the axis 116, such that the needle bearing 325, bushing 330 and header 340 travel along the longitudinal axis 316 and spin or rotate three-hundred sixty degrees about the axis 316 simultaneously together with one another about the pivot 335 without applying rotational or bending torque or strain on the stem 314 of the sensor 312. This construction reduces undesired torque on the sensor 312.

The header 340 is also operable to swivel simultaneously with the needle bearing 325 and bushing 330 about the ball pivot 335 with respect to the housing 310, so as to move or deflect an angular deflection or distance (θ) relative to a generally vertically aligned position. Accordingly, the header 340 can move or deflect in any direction (θ) about a three-hundred sixty degree plane aligned generally perpendicular to vertical, which is shown generally coincident with the central longitudinal axis 316 of the control header assembly 300. With deflection of the header 340 in any angular direction (θ) from the axis 316, a force is transferred via the needle bearing 325 and the lever 380 to the stem 314 of the sensor 312. The strain gauges 320 and 322 generate an electrical signal representative of the measured direction and force or strain associated with bending the stem 314 of the sensor 312. The sensor 312 communicates the electrical signal to the controller 305 (See FIG. 2). The bushing 330 is configured as a mechanical stop to limit a lateral deflection of the header 340 away from the axis 316, as well as retain or limit longitudinal deflection of the header 340 away from the control handle assembly 300 along the axis 316, so as to reduce opportunities of excessive force that can damage the sensor 312. The housing 310 generally includes a limit 355 to retain the bushing 330 and header 340 in construction with the control handle assembly 300.

The header 340, needle bearing 325 and bushing 330 also move together in a generally co-linear or vertical direction along the central longitudinal axis 316 of the control handle assembly 300. A spring 350 is generally centrally located along the axis 316 to receive the needle bearing 325, bushing 330 and structural legs 342, 344 therethrough. A spring 500 is generally located against the housing 310 in a manner that biases the header 340 in an upwardly or outwardly direction relative thereto as well as with respect to the pivot 335 or the sensor 312 mounted therein. Another spring 505 is generally located within the legs of the 342 and 344 of the header 340 and receives a portion of the header 340 therethrough to generally hold the spring 505 in place relative to the remainder of the control handle assembly 300. The spring 505 also generally applies a bias force in outward direction, similar to the spring 500, so as cause a predetermined force against the header 340 to be overcome in depressing the header 340 to active the control handle assembly 300 to transmit signals to the controller 305.

Accordingly, the header 340 is configured such that an operator can depress or compress or actuate the header 340 from the generally upward, inactive position simultaneously with deflection or movement of the header 340 in a generally horizontal or lateral or perpendicular direction relative thereto. Movement of the header 340 in the general longitudinal direction 144 opens and closes an electrical pathway or switch 510 such that electrical signals can transmit from the sensor 312 to the controller 305.

For example, the biased upwardly or outwardly position of the header 340 relative to the housing 310 or pivot 335 interrupts communication of the electrical signals from the sensor 312 to the controller 305. Compressing or moving the header 340 to a lowered, active position relative to the housing 310 or pivot 345 or sensor 312 closes an electrical path or switch such that the electrical signals from the sensor 312 are operable to be communicated to the controller 305.

Referring to FIGS. 1 and 2, one embodiment of the power assist system 120 includes the first control handle assembly 300 located at the table 270 and connected via the controller 305 in communication with one or more of the drive mechanisms 272, 276, 278 to move tabletop 274, as described above. The power assist system 120 also includes a second control handle assembly 405 located at the elbow 150 and connected via the controller 305 in communication with one or more of the drive mechanisms 180, 200, 245 or 255 to move mobile arm 145, as described above. Of course, the power assist system 120 can include only one if the first and second control handle assemblies 300 and 405, as described above, or additional control handle assemblies located to move other medical equipment, or be attached at the other mobile components (e.g., mobile arm 145) different than that shown.

Having generally provided the above-description of a construction of the embodiment the system 100, the following is a general description of a method 200 of operation of the system 100 in acquiring variable directional medical image data of a subject 110. It should also be understood that the sequence or succession of the acts or steps of the method 200 as described in the foregoing description can vary. Also, it should be understood that the method 200 may not require each act or step in the foregoing description, or may include additional acts or steps not disclosed herein. One or more of following steps and acts of the method 200 can also be in the form of computer-readable program instructions for execution by the controller 305 or other programmable device.

Assume initially that the support assembly 140 of the imaging system 115, as well as the tabletop 274 of the table 270, is at a parked or zero or stowed position, and the subject 110 is located at the table 270 to be imaged by the imaging system 115. Both the first and second control handle assemblies 300 and 405 are generally vertically aligned with the respective central longitudinal axis 316 of the assembly 300 or 405. The header 340 of each control handle assembly 300 and 405 is biased to an extended, inactive position such that electrical signals from the respective control handle assemblies 300 and 405 are interrupted from communication that might otherwise trigger or cause the respective drive mechanisms 180, 200, 245, 255, 272, 276, or 278 to move the tabletop 274 or the support assembly 140, respectively.

At the table 270, an operator applies a force so as to compress or depress the header 340 relative to the bushing 330 so as to enable electrical signals from the control handle assembly 300 to trigger movement of at least one of the drive mechanisms 272, 276, 278 at the table 270. With the header 340 compressed, assume the operator also moves or deflects the header 340 of the control handle assembly 300 in a direction of desired movement of the tabletop 274 of the table 270. The force to laterally deflect the header in a direction is at least partially communicated to the stem 314 of the sensor 312. The sensor 312 of the control handle assembly 300 detects and translates deflection of the stem 314 associated with or caused by deflection of the header 340 into an electrical signal representative of an instruction to move the tabletop 274 in the detected direction of deflection of the header 340. The sensor 312 is operable to detect any deflection of the header in a three-hundred sixty degree plane relative to the generally vertical, central longitudinal axis 316 of the control handle assembly 300.

The construction of the ball pivot 335 in combination with the sensor 312 and header 340 allows the control handle assembly 300 to steer the tabletop 274 or gantry/support assembly 140 similar in manner to a joystick. Moving the control handle assembly 300 to steer movement of the tabletop 274 in this joystick manner of operation about the ball pivot 335 with respect to the table 270 or tabletop 274 reduces the risk of causing simultaneously movement of the table 270. The construction of the ball pivot 335 also reduces opportunities of causing damage to the sensor 312. With the described construction of the ball pivot 335 in combination with the stem 314 and strain gauges 320, 322, the power assist system 120 can also include a circuit to amplify the signal generated by the sensor 312, thereby reducing the force to be exerted on the header 340 and stem 314 to generate the signal to steer the tabletop 274 or gantry/support assembly 140. Another way to amplify the signal is to adjust a distance between the header 340 and the point 338, or from the point 338 to the sensor 312. For example, with decreasing the distance between the header 340 relative to the point 338 and increasing the distance from the point to the sensor 312, a small displacement or movement of the header 340 can cause a larger displacement or strain at the sensor 312, causing generation of a larger signal from the sensor 312 to the controller 305.

As long as the header 340 of the control handle assembly 300 is in the lowered or depressed active position, then the controller 305 receives the electrical signal from the sensor 312. The controller 305 translates the electrical signal from the sensor 312 to a control signal representative of instructions to the one or more drive mechanisms 272, 276, 278 to move the tabletop 274 in the direction as directed by the operator via the control handle assembly 300. For example, if the direction of the header 340 as represented by the electrical signal is representative of a lateral direction, then the controller 305 calculates and generates a control signal generally to instruct the one or more drive mechanisms 272 to the move the tabletop 274 in the lateral direction.

In another example, if the direction of the header 340 as represented by the electrical signal is representative of a generally diagonal direction between lateral and longitudinal directions along the horizontal or x-y plane, then the controller 305 calculates and generates a single or independent control signal to drive mechanisms 272 and 276 to move the tabletop 274 in both the lateral and longitudinal direction (e.g., simultaneously or intermittently) so to result in movement of the tabletop 274 in the detected diagonal direction. Of course, a single drive mechanism may be coupled to move the tabletop 274 in any direction along the generally horizontal plane in place of the dual drive mechanisms 272 and 276. Also, the control handle assembly 300 can be configured, aligned or located such that the sensor 312 of the control handle assembly 300 can detect movement or deflection of the header 340 along a vertical or x-z or y-z plane, and correspondingly generate an electrical signal representative of the direction in the vertical plane for communication to the controller 305. In response, the controller 305 generates a control signal to instruct one or more drive mechanisms 272, 276 and 278 to move the tabletop 274 in the direction vertical plane, in the general direction of the header 340.

Upon release of the header 340 from the depressed, lowered or active position, the spring 350 biases the header 340 to the raised, inactive position, such that the bushing 330 is generally biased against the housing 310 and restrains the header 340. The raised, inactive position of the header 340 interrupts communication of the electrical signal from the control handle assembly 300 to the controller 305. As a result, movement of the header 340 in generally horizontal plane or direction does not cause communication of the control signal from the controller 305 to the one or more drive mechanisms 272, 276, 278 and correlated movement of the tabletop 274 in the respective detected direction of the header 340.

In a similar manner, movement of the second control handle assembly 405 at the gantry or support assembly 140 regulates movement of the elbow 150 and mobile arm 145 with respect to the imaged subject 110. For example, an operator applies a force so as to compress or depress the header 340 toward the bushing 330, enabling electrical signals from the control handle assembly 405 to trigger movement of one or more of the drive mechanisms 180, 200, 245, 255 at the gantry 140. With the header 340 compressed, assume the operator moves or deflects the header 340 of the control handle assembly 405 in a direction of desired movement of the mobile arm 145. The sensor 312 of the control handle assembly 405 detects and translates deflection of the stem 314, associated with or caused by deflection of the header 340, into an electrical signal representative of an instruction to move the mobile arm 145 in the direction of detected deflection of the header 340.

As long as the header 340 of the control handle assembly 405 is in the lowered or depressed active position, then the controller 305 receives the electrical signal from the sensor 312. The controller 305 translates the electrical signal from the sensor 312 to a control signal representative of instructions to the one or more drive mechanisms 180, 200, 245, 255 to move the mobile arm 145 in the direction as directed by the operator via the control handle assembly 405. The controller 305 calculates and generates control signals generally to instruct the one or more of the drive mechanisms 180, 200, 245, 255 to the move the mobile arm 145 generally in the direction of the header 340 from reference. The control signal can cause intermittent or simultaneous drive operation of the drive mechanisms 180, 200, 245, 255 so to result in movement of the mobile arm 145 in the selective direction as input via the control handle assembly 405.

A technical effect of the above-described system 100 and method includes an imaging system 115 in combination with a power assist system 120 with generally enhanced mobility that can be readily put in arbitrary positions in a crowded work environment. The power assist system 120 operable to change a position of the imaged subject 110 with respect to the image detector 135 in a smooth manner with generally less measured effort applied by an operator. A kill or enable mechanism (i.e., compression of header 340 to close electrical pathway for communication of sensor signals to the controller 305) of the power assist system reduces the likelihood of uncontrolled motion of the subject 110 or image detector 135 with respect to one another. The power assist system 120 is also configured so as to reduce a likelihood of excessive mechanical torque or force from causing damage to the sensor 312 of the control handle assembly 300 or 405.

Although the above-described system 100 is directed to medical imaging, the system 100 is not so limited. The system 100 and method is also applicable to industrial imaging of a subject 110, imaging directed to security, etc. and is hereby considered within the subject matter described herein.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of assisting movement of a tabletop in support of a subject to be imaged with an image acquisition system, the method comprising the acts of:
detecting and generating an electrical signal representative of a direction of deflection of header of a control handle assembly coupled to the tabletop;
interrupting communication of the electrical signal to a controller;
communicating the electrical signal to a controller in response to detecting compression of the header of the control handle assembly;
generating a control signal at the controller to instruct at least one of a first and a second drive to move the tabletop in the direction of deflection of the header, and
moving the tabletop in the direction of deflection of the header of the control handle assembly via the at least one of the first and second drives in accordance to the control signal.

2. The method of claim 1, further including the act of:
biasing the header toward an outward position so as to cause the interrupting step.

3. The method of claim 1, wherein the act of detecting the electrical signal representative of the direction of deflection of the header of the control handle assembly includes measuring a direction of force on a step via generally perpendicularly aligned first and second strain gauges.

4. The method of claim 1, the method further comprising the acts of:
slidably coupling the stem of the sensor to a first end of a bushing; and
pivotally supporting the bushing in a housing.

5. The method of claim 4, the method further comprising the acts of:
slidably coupling the second end of the bushing to the header, the second end opposite the first end.

6. The method of claim 1, the method further comprising the acts of:
limiting deflection of the header below a threshold to damage to the sensor.

7. The method of claim 1, the method further comprising the acts of:
providing a third drive to move the tabletop to an angular alignment different from generally horizontal; and
generating a control signal to instruct at least the third drive to move the angular alignment of the tabletop in the direction of deflection of the header.

8. A system having a radiation source and a detector operable to generate a radiological image of a subject from multiple directions, the subject supported on a tabletop of a table from a floor, the system comprising:
a first drive operable to move the tabletop in a first generally horizontal direction;
a second drive operable to move the tabletop in a second generally horizontal direction generally perpendicular to the first horizontal direction;
a control handle assembly coupled to the table, the control handle including a sensor operable to generate an electrical signal representative of a direction of deflection of a handle from a neutral position; and
a controller in communication with the first and second drives and the control handle assembly, the controller including a processor in communication to execute a plurality of program instructions stored in a memory, the plurality of programs instructions representative of the steps including:
translating the electrical signal from the sensor into the direction of deflection of the control handle,
generating a control signal to instruct at least one of the first and second drives to move the tabletop in the direction of deflection, and
moving the tabletop in the direction of deflection of the control handle assembly via the at least one of the first and second drives in accordance to the control signal.

9. The system of claim 8, further comprising a third drive operable to move the tabletop in a generally vertical direction, the control handle assembly and the controller in communication with the third drive, wherein the step of generating the control signal is to instruct one of the first, second and third drive to move the tabletop in the direction of deflection in the vertical direction.

10. The system of claim 8, wherein the sensor comprises first and second strain gauges, the first strain gauge operable to measure a force of deflection of a stem from a reference in a first direction, and the second strain gauge operable to measure a force of deflection of the stem from the reference in a second direction generally perpendicular to the first direction.

11. The system of claim 10, wherein one end of the sensor is slidingly coupled to a first end of a needle bearing pivotally supported by a pivot mounted in a housing of the control handle assembly, wherein the needle bearing travels along a longitudinal axis of the sensor, and spins three-hundred sixty degrees about the longitudinal axis without applying bending strain on the sensor.

12. The system of claim 11, wherein the second end of the needle bearing is sliding coupled to a header, the second end opposite the first end.

13. The system of claim 12, wherein the header moves between a compressed position toward the pivot and an extended, biased position outward from the pivot, wherein the header in the compressed position closes an electrical pathway to communicate the electrical signal from the sensor to the controller, and wherein the header in the second, outward position interrupts communication of the electrical signal to the controller.

14. The system of claim 13, wherein a second end of the sensor is fixed relative to the housing.

15. A system having a radiation source and a detector operable to generate a radiological image of a subject from multiple directions, the source and detector supported from a gantry, the system comprising:
a first drive coupled to move the gantry and the detector about a generally vertically aligned first axis;
a second drive coupled to move the gantry and the detector about a generally horizontal aligned axis;
a control handle assembly coupled to the gantry in mobile support of the detector, the control handle assembly including a sensor operable to generate an electrical signal representative of a direction of deflection of a header from a neutral position; and
a controller in communication with the first, second and third drives and the control handle assembly, the controller including a processor in communication to execute a plurality of program instructions stored in a memory, the plurality of program instructions representative of the steps including:
translating the electrical signal from the sensor into the direction of deflection of the header,
generating a control signal to instruct at least one of the first and second drives to move the gantry in the direction of deflection, and
moving the gantry in the direction of deflection of the header via the at least one of the first and second drives in accordance to the control signal.

16. The system of claim 15, further comprising:
a third drive coupled to move the gantry and the detector about a generally vertically aligned second axis spaced an offset distance from the first axis,
wherein the control handle assembly and the controller are connected in communication with the third drive, wherein the step of generating the control signal is to instruct one of the first, second and third drives to move the gantry and the detector in the direction of deflection of the header of the control handle assembly.

17. The system of claim 15, wherein the sensor comprises first and second strain gauges, the first strain gauge operable to measure a force of deflection of a stem from a reference in a first direction, and the second strain gauge operable to measure a force of deflection of the stem from the reference in a second direction generally perpendicular to the first direction.

18. The system of claim 15, wherein one end of the sensor is slidingly coupled to a first end of a needle bearing pivotally supported by a pivot mounted in a housing of the control handle assembly, wherein the needle bearing travels along a longitudinal axis of the sensor and spins three-hundred sixty degrees about the longitudinal axis without applying bending strain on the sensor.

19. The system of claim 18, wherein the second end of the needle bearing is coupled to the header, the second end opposite the first end.

20. The system of claim 19, wherein the header moves between a compressed position toward the pivot and an extended, biased position outward from the pivot, wherein the header in the compressed position closes an electrical pathway to communicate the electrical signal from the sensor to the controller, and wherein the header in the second, outward position interrupts communication of the electrical signal to the controller.
